(19) United States
(12) Patent Application Publication
(10) Pub. No.: US 2019/0267117 A1
(43) Pub. Date: Aug. 29, 2019

(54) OTITIS MEDIA PREVENTION SYSTEM

(71) Applicants: Christine Tinio Geronimo-Button, Manorville, NY (US); Terry Michael Button, Manorville, NY (US); Ashish Verma, Port Jefferson, NY (US)

(72) Inventors: Christine Tinio Geronimo-Button, Manorville, NY (US); Terry Michael Button, Manorville, NY (US); Ashish Verma, Port Jefferson, NY (US)

(21) Appl. No.: 16/411,083

(22) Filed: May 13, 2019

Related U.S. Application Data
(63) Continuation of application No. 14/994,190, filed on Jan. 13, 2016.

(51) Int. Cl. G16H 10/20; G16H 40/63; G16H 10/60

(52) U.S. Cl. CPC G16H 10/20; G16H 10/60; G16H 40/63

(57) ABSTRACT

In an embodiment, the present invention provides a computer system for use by caregivers in the prevention and treatment of acute otitis media (AOM) with the use of the Galbreath technique in children from shortly after birth to about six years of age. The computer system assists caregivers in the monitoring of children who may be at risk for AOM or who may be experiencing an active episode of AOM. The system may make automated suggestions in real time of treatment options available to a caregiver at home. The computer system may include a diary component, and may include a Virtual Coach component providing reminders in real time and educational materials in the use of the Galbreath Technique and other medical interventions in the prevention and treatment of AOM.

### CROSS-REFERENCE TO RELATED APPLICATIONS

This application is a continuation of U.S. patent application Ser. No. 14/994,190 filed Jan. 14, 2016, which claims priority to U.S. patent application Ser. No. 62/103,323, filed Jan. 14, 2014.

### BACKGROUND OF THE INVENTION

Acute otitis media (AOM) is the most common illness in infants and children with 75-80% of the U.S. population having an episode by age 3. AOM (ear infections) are the most common infection for which antibiotics are prescribed for children in the U.S. Hearing deficits, speech delay and learning disorders can occur if ear infections become chronic. The estimated medical costs and lost wages because of ear infection approach $5 billion per year in the U.S. However, based on the relationship between audition, language and learning, children with a significant history of ear infection and subsequent auditory deficits may be delayed in linguistic and cognitive development. This puts the cost of ear infection much higher.

Treatment of AOM is a controversial subject because it is difficult to distinguish viral from bacterial infections. Viral based otitis media usually resolves without antibiotics. Acute bacterial, otitis media, however, can cause eardrum perforation and can spread to cause mastoiditis and/or meningitis, brain abscess, and even death if a severe infection goes untreated long enough. In addition, high fever can cause febrile seizures. Antibiotics prevent most such complications, but have also led to the inappropriate use of antibiotics and subsequent resistance. Rational approaches have included deferring the start of antibiotics for one to three days. This reduces the use of antibiotics in two of three children without adverse outcomes, but may prolong the illness in some patients.

Otitis media is an inflammation or infection of the middle ear. Otitis media is very common in young children typically as the result of an upper respiratory infection spreading to the ear. The pathophysiology is usually attributed to a dysfunction of the Eustachian tube that can occur in young children, up to the age of about 6 years. The normal Eustachian tube is usually closed but opens regularly to replenish the air in the middle ear. However, a Eustachian tube that is blocked by swelling or plugged with mucus from a cold cannot open to ventilate the middle ear. This lack of ventilation may allow fluid to accumulate. This fluid is a fertile media for infection to grow.

The peak ages for occurrence of AOM are children aged 3-18 months. (http://emedicine.medscape.com/article/859316-overview#a7, checked Dec. 28, 2015). The incidence occurs as early as shortly after birth, and declines after age 18 months.

Age based variations of the Eustachian tube may be an underlying factor for the predominance of otitis media in young children. For example, the adult Eustachian tube has a cross-sectional area 2.5 times larger than typical in a 2-year-old child. In addition, the Eustachian tube is more horizontal and less compliant in children due to sparse amounts of elastin when compared to adults. It has also been suggested that as the human body develops, the muscles that open the Eustachian tube increase in size, enhancing their ability to open the tube itself.

As a result of these differences, fluid can become more easily trapped in the middle ear of a young child causing upper respiratory infections.

A simple treatment for otitis media was developed and reported in 1929 by William Otis Galbreath, D.O. The Galbreath method involves applying simple mechanical methods to promote natural fluid drainage. It has been suggested that physicians can easily teach this procedure to parents for use at home. (D. Pratt-Harrington, Galbreath technique: a manipulative treatment for otitis media revisited. The Journal of the American Osteopathic Association, October 2000, Vol. 100, 635-639).

In the Galbreath technique, the patient is either supine or sitting on the caregiver's lap. The caregiver turns the child's head so that the affected ear faces away. The caregiver uses the hand that is opposite of the affected ear to provide treatment. For treatment, the caregiver contacts the child's mandible on the affected side and applies a downward and transverse mild force on the mandible that crosses the face. This is repeated in a slow rhythmic motion (about 3 to 5 seconds per round) for 30 to 60 seconds. The technique involves simple facial manipulation (massage like action) designed to drain the estuation tube.

Clearly, prevention of infection is ideal. Considerable effort has been undertaken in the development of an otitis media vaccine, however such vaccines have had limited success, reducing the number of episodes of acute otitis media by 34 percent. A recent review pneumococcal conjugate vaccines found no benefit in preventing acute otitis media infection. (Fortanier, “Pneumococcal conjugate vaccines for preventing otitis media.” Cochrane Database of Systematic Reviews 2014 (4), CD001480). A more natural approach such as the Galbreath technique, however, would seem advantageous. If proven to match preliminary reports in the literature of 70% effectiveness, the Galbreath technique would be superior to vaccines.

Numerous reports have been made in the literature demonstrating the potential of osteopathic manipulation in the treatment and prevention of otitis media. A study by Steele showed 6 of 9 patients with acute otitis media were successfully treated by osteopathic manipulative treatment (J Am Osteopath Assoc. 2014; 114(6):436-447). Degenhardt and Kuchera reported the results of three weeks of osteopathic intervention on a small group of patients with recurrent otitis media (J Am Osteopath Assoc. 2006; 106:327-334). Five of eight benefited and half had resolution of their symptoms for at least a one year after treatment. While it is difficult to draw conclusions from a small population without randomized controls, the results are encouraging. A larger group of 57 subjects were studied of which 25 had three weeks of osteopathic intervention and routine medical care and 32 controls had only routine medical care. (Mills, Arch Pediatr Adoles Med, 2003, 157 (September 2003) 861). The patients treated with osteopathic manipulation had fewer episodes of acute otitis media, required fewer surgical procedures and had more mean surgery-free months than those without osteopathic manipulation. In addition, baseline and final tympanograms obtained by an audiologist showed an increased frequency of more normal tympanogram types in those treated by osteopathic manipulation.

Overall, the medical evidence appears to show that the Galbreath technique is a safe and effective treatment for AOM that can be taught to parents and caregivers, and may avert the use of antibiotics in many cases. Furthermore, the evidence appears to show that the Galbreath technique can be applied prophylactically to children at risk for AOM, or who have a significant history of AOM, and this use prevents the likelihood of an occurrence of AOM in such children. Moreover, the Galbreath technique is also effective if applied during upper respiratory infections.

### SUMMARY OF THE INVENTION

A problem with the use of the Galbreath technique (as well as other medical interventions) is that caregivers may fail to administer the technique on a sufficiently timely basis, and there is a need for methods and systems to remind caregivers to perform the technique. For example, in certain patients, it may be advantageous to administer the Galbreath technique two times, three times, four times, five times, or six times per day (or even more frequently). As a general proposition, it is typically advantageous to administer the technique at evenly spaced intervals, for example every six hours if the technique is to be administered four times per day. Even if the technique is prescribed once per day, it is typically advantageous to administer it at the same time each day. Furthermore, as the disease progresses in any particular child, it may be advantageous to vary the frequency of administration of the technique. For example, a physician may prescribe using the technique every four hours for one day, then every six hours for the following two days, then every 12 hours for the following seven days. Optimal treatment requires that the technique be administered as prescribed, and for many caregivers, reminders based on the physician recommendation can be very helpful.

The instant invention is designed to solve this problem. The inventive system may be able to incorporate physician instructions and provide reminders so that caregivers may be reminded in real time to administer the Galbreath technique to a child at specific times, for example, 8 AM and 8 PM. The reminders may be delivered by mobile notification message, short message service (SMS) text message, email, or an application specific vibration or audible alarm on a mobile device.

Accordingly, a data-enabled apparatus and method is provided to assist parents and caregivers in monitoring AOM incidences and assist caregivers in the use of the Galbreath technique to prevent and treat AOM in children up to about age 6, the peak years for AOM. The apparatus may be implemented on a handheld computer such as a smartphone or tablet, or it may be implemented on a laptop or desktop computer, or it may be implemented through a website.

The apparatus and method includes a software application, hereinafter termed a “Otitis Media Prevention Module” (OMPM), consisting of a computer application, running on computers such as Mac, Windows, iOS, Android, or as a web service. Coupled to the OMPM is a Records Management Database (RMDB) that stores data entered by the user. The RMDB may also include pre-loaded data such as demographic and standardized medical data.

This invention may provide educational tools including an interactive diary and a virtual coach. Educational components will provide caregivers the requisite background in ear infection prevention and describe how to use the Module tools. The diary allows the caregiver to track the child's upper respiratory health and action taken. The virtual coach minds users to check and record their child's upper respiratory health and if an upper respiratory infection is beginning, reminds the caregiver to provide treatment and record in their diary their child's ear health. With these tools, the Galbreath technique may be applied routinely to dramatically reduce the occurrence of otitis media.

In an embodiment, the OMPM contains a diary wherein a caregiver can record relevant data about the child, such as body temperature, degree of nasal discharge, appearance of pain, mood, and other factors relevant to AOM.

In an embodiment, the OMPM may prompt the caregiver with questions that may be posed at specific times of the day (such as 8 AM and 8 PM). The questions are directed to factors pertaining to ear health that can be entered into the diary, such as body temperature, degree of nasal discharge, appearance of pain, mood, and other factors relevant to AOM.

In an embodiment, the OMPM may provide instructional material pertaining to ear health, such as dietary modifications to reduce mucus production, use of humidifiers, and administration of medications (prescription and non-prescription). The OMPM may also contain instructional information on the Galbreath Technique, including how to apply the technique. This is an adjunct to the instructions to the caregiver that should be provided by a health care professional on how to administer the Galbreath Technique to their child.

In an embodiment, the OMPM may include a virtual coach that reminds the caregiver to make specific observations of the child's health, to record those observations in the diary component, and to administer medical interventions. The medical interventions can include directions to apply of the Galbreath Technique, administration of medications, use of humidifiers, and dietary modifications. For directions such as application of the Galbreath Technique, administration of medications, use of humidifiers, where timing is important, the Virtual Coach may provide real time reminders, for example, a reminder at 8 PM that the caregiver should apply the Galbreath Technique at that time.

### BRIEF DESCRIPTION OF THE DRAWINGS

FIG. 1 is an embodiment of a diary page.

FIG. 2 is an embodiment of an expanded diary page.

### DETAILED DESCRIPTION

As used herein, the term “computer” refers to any device having a microprocessor capable of running the software of the invention. The term may refer, for example, to a handheld device such as a smartphone or tablet computer, which may be termed a “mobile computer,” or a notebook computer, laptop computer, or desktop computer, which collectively may be termed a “personal computer.” The terms “patient” and “child” are used interchangeably, unless the context indicates that a distinction is being made. The term “caregiver” refers to a parent of a child, and also to an adult who cares for the child, and includes persons such as relatives, nannies, teachers, day-care workers, and nurses or other medical personnel. The terms “parent” and “caregiver” are used interchangeably herein.

In an embodiment, the present invention provides a computer system and software application, hereinafter termed a “Otitis Media Prevention Module” (OMPM), for use by caregivers in the prevention and treatment of acute otitis media (AOM) with the use of the Galbreath technique in children from shortly after birth to about six years of age. The computer system assists caregivers in the monitoring of children who may be at risk for AOM or who may be experiencing an active episode of AOM. The OMPM may include a diary component, and may include a Virtual Coach component providing reminders and educational materials in the use of the Galbreath Technique and other medical interventions in the prevention and treatment of AOM.

In an embodiment, the OMPM may be implemented on several computer platforms, and users can employ any one of the platforms or a combination thereof. For example, the OMPM may be implemented on a mobile platform, such as an iOS® or Android™ smartphone, a wearable computer (such as a smart watch or other jewelry), or a tablet computer. As used in this disclosure, the term “mobile app” refers to such a use.

The OMPM may also be implemented on a desktop or laptop-type computer such as on computers running the Windows® or Macintosh® operating systems. Desktop or laptop-type computers are also referred to herein as “personal computers.” As used in this disclosure, the term “desktop app” refers to the OMPM implemented on a desktop or laptop computer. The OMPM may also be implemented on a website. As used in this disclosure, the term “website” refers to such a use.

In addition to the OMPM, a user website may be provided, that will contain most or all of the tools in the users personal OMPM, and the user will communicate with the OMPM through the internet. The website also may be configured to gather data, either personally identifiable or anonymized, for example for data aggregation purposes. Data stored by mobile apps, desktop apps, and/or websites may be synchronized so as to permit a patient to run the OMPM on more than one platform.

In an embodiment, a computer system (OMPM) is provided for use by a user, wherein the user is a caregiver of a child, wherein the child is at risk for an upper respiratory infection, at risk for otitis media or the child is suffering from an upper respiratory infection or otitis media. The system may have a computer, a user interface operating on the computer, the user interface having a display screen and keyboard for text input, a records management database stored in a non-volatile electronic medium operatively coupled to the computer, and a data management application coupled to the user interface and running on the computer. The records management database may store data relevant to otitis media in the child, and a diary of data entered by the user. The user may enter relevant data into the records management database via the user interface on the initiative of the caregiver or in response to questions generated by the data management application. The data management application may be preprogrammed with information on the etiology and treatment of otitis media. The data management application may be programmed to process data in the records management database and deliver pre-programmed messages or questions to the patient in real time. The messages and questions may instruct or assist the caregiver in real time and provide information on lifestyle choices and therapeutic modalities that can be used by the caregiver for the prevention or treatment of otitis media in the child.

In an embodiment, the OMPM computer system may be implemented on a computing platform selected from a website, a smartphone, a tablet computer, a desktop or laptop computer, and a wearable computer, or a combination thereof. For example, there may be an embodiment in which the database is on a remote network-accessible server, and the user can use the database on a smartphone embodiment, a desktop embodiment, and a website embodiment. The system may provide reminders, video clips, the diary component on any platform on which it is implemented.

The keyboard of the inventive system may include a virtual keyboard or a physical keyboard in a conventional “QWERTY” layout, or some other arrangement of keys. The term “keyboard” also includes at least one key that can be pushed to convey user choices to the computer. For example, simple wearable devices may have a small number of buttons that allow the user to scroll through various choices and make a selection.

The reminders of this invention may be in the form of email messages, mobile notification message, SMS text messages, application specific text messages, for example, a pop up dialog within an application embodiment of this invention, vibrations, and alarm tones, or any combination thereof. The reminders are used to inform the caregiver of a task, such as administering a medical intervention, making an observation, or making a diary entry, wherein the task is time sensitive. Thus, in an embodiment, the reminders are delivered in real time. For example, there may be an instruction to perform the Galbreath technique at 8 PM. The inventive system would issue a reminder at 8 PM to perform the technique on the patient.

The OMPM may include a records management database (RMDB). This may be any type of database system conventionally known in the art, such as a relational or flat database that stores an organized collection of data in non-volatile computer memory. The database may be stored locally on a user's device such as a mobile or desktop computer. Alternatively, the database may be may be stored at a remote location and is accessed via a network connection.

The database may store data such as caregiver and patient basic data such as name, birthdate, role, and login credentials. The database may also store the data used in the diary component of this invention. The database may also physician instructions. The database may store generic medical information relevant to otitis media, such as commonly used drugs, medical discussions of the causes and symptoms of otitis media, standard system messages, and other educational information relevant to otitis media.

The inventive system may also include a data management application (DMA) component, that interfaces with the RMDB and is programmed to perform tasks such as reminding the user to perform certain tasks at certain times, to solicit input from the user in the form of queries, to send messages to the user in real time with recommendations and advice, and other tasks.

In various embodiments, the DMA may perform any or all of the following tasks:
it incorporates personal details of the child, such as name, birthdate, and sex, and includes medical details such as height and weight, history of upper respiratory infection and/or otitis media, and any other significant medical issues;
it may pose questions to the user intended to solicit input for the diary;
it provides an interface for the diary features of the invention;
it prescribes the frequency the Galbreath technique should be performed on the child;
it makes suggestions regarding the when and how often the Galbreath technique should be performed based on information on the age and health status;
it issues instructions to the user, for example, to perform the Galbreath technique at a certain time of day;
it issues instructions in real time to a user, for example, it sends a reminder at 6 PM that it is time to perform the Galbreath technique at that time; and
it provides educational materials to the user, for example, on the nature otitis media and how the Galbreath technique is performed.

In an embodiment, the educational material on the Galbreath technique may include a video clip.

In an embodiment, the inventive system may include a method permitting the caregiver to ask their physician a question, and communicate directly with the physician.

In an embodiment, the inventive computer system will have conventional security features such as a username and password to secure its access. This is necessary to prevent unauthorized access and hacking of the system. In addition, data transmission encryption methods may be used, particularly since (in some embodiments) the data may be transmitted across public networks. Such data transmitted on public networks may be encrypted using standard encryption protocols like https, SSL/TLS, AES or other standards known in the art or that may be developed in the future.

In an embodiment, the OMPM will have an initial phase of operation in which the caregiver enters relevant details about the patient (the child), such as name, sex, and birthdate. In addition, during the initial phase of operation, relevant background material will be collected, such as name of the child's physician, relevant medical history including a history of upper respiratory infection and otitis media.

In an embodiment, the OMPM will be used for a single patient (child). In another embodiment, the OMPM system may have discrete accounts or discreet RMDB instances for a plurality of children. In an embodiment, the OMPM may permit a caregiver to manage several children at the same time, for example by having different accounts for each child. Each account will have an RMDB that is unique to that child. This multiple account embodiment may be useful in clinical or institutional settings, such as hospitals, schools, daycare settings, or even parents with twins, triplets, or children close in age that are prone to upper respiratory infection or otitis media.

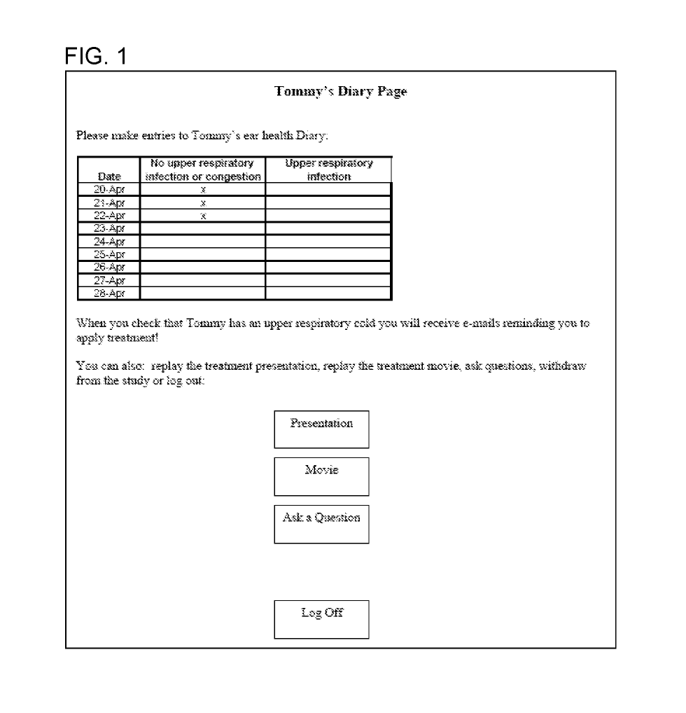

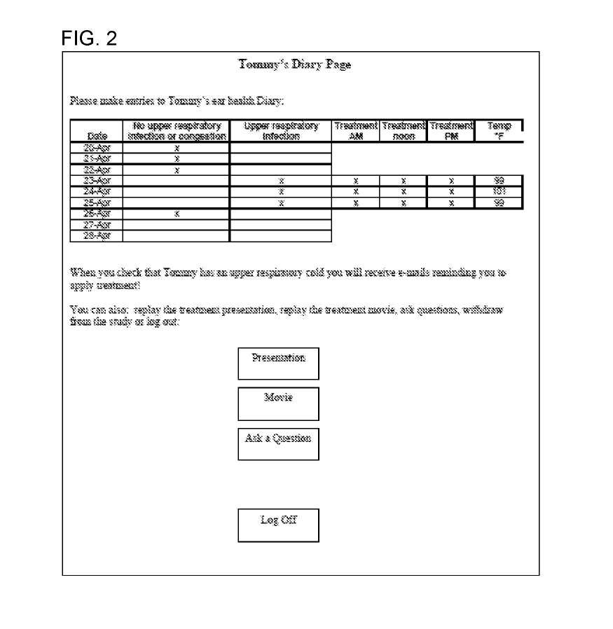

In an embodiment, an example of a diary page is shown in FIG. 1. This figure shows a table of data and various buttons for options that the system can perform, such as viewing educational materials. FIG. 1 shows an embodiment of an interactive diary used to track a child's ear heath. Reminder notifications may be sent to the caregiver twice each day to document the wellness of the child under their care. Typically, the response will be to select “No upper respiratory infection or congestion.” Upon entry of an upper respiratory episode, however, the diary expands as shown in FIG. 2. This expanded form shows, for example, fields for documenting medical data such as body temperature, and includes a schedule of when the caregiver should apply Galbreath technique. Reminder mobile notifications may also be sent to the caregiver each day that an upper respiratory event is recorded. Should the child develop ear pain, or a fever that exceeds 102° F., the caregiver will be instructed to take the child to their physician promptly.

The function keys shown on the diary in FIGS. 1 and 2 show, for example, options to view a power point presentation (educational material), view a video clip demonstrating the use of treatment technique or ask questions. Because of the educational materials, memorizing the treatment details is not required. The caregiver can refresh their skills as needed.

In an exemplary operation, each day the caregiver checks the “Upper respiratory infection” box, the process discussed above, with the expanded diary will be shown. When “No upper respiratory or congestion” is selected following an upper respiratory infection, a modified diary will prompt the caregiver to enter “yes” or “no” to the following questions:

1. Did your child require a visit to the physician? (If yes—next question; if no—stop)

2. Did the physician confirm an ear infection? (If yes—next question; if no—stop)

3. Were antibiotics used? (If yes—next question; if no—stop)

4. Were myringotomy tubes required?

In an embodiment, the information observed by the caregiver and entered into the diary may be transmitted directly to a physician or other medical professional, allowing the physician to monitor remotely the status of the patient. In an embodiment where the OMPM communicates directly with a physician or other medical professional, the medical professional is in a position to respond in real time with treatment options that can be performed by the caregiver, such as performing the Galbreath technique, administering medications, or recommending other treatments such as lifestyle changes.

In an embodiment, the OMPM is useful to treat AOM. The system is used to gather information on the patient, and to issue instructions to the caregiver to perform medical interventions expected to helpful in treating AOM. In an embodiment, the inventive system may issue recommendations, reminders, and instructions in real time to perform a Galbreath technique on a child at a specific time. The system may also issue real time reminders to administer drugs, such as analgesics, decongestants, and antibiotics.

In an embodiment, the instant invention is useful to prevent otitis media infections. By the monitoring the ear health of children, particularly those at risk for otitis media, medical professionals and caregivers may be able to take remedial measures that avert the occurrence of otitis media infections. Such remedial measures may include scheduling the Galbreath technique, for example once per day, or several times per week as a prophylactic intervention. The inventive system may prompt caregivers to perform that the Galbreath technique on such a schedule. In addition, the system and diary component records medical data such as body temperature, appearance of nasal discharge, and other factors of interest in otitis media that can be used to monitor and record the health of the child.

Diet has also been implicated in the prevention and treatment of otitis media. (Green, Can Fam Physician, 1983 January; 29: 15; James, Curr Allergy Asthma Rep. 2004 July; 4(4):294-301 (abstract only)). For example, it has been suggested that sugary drinks, milk, cheese, and cereal grains can cause otitis media or exacerbate symptoms, and removal of these food items for even a few days can have a very beneficial effect. The OMPM can track the use of such food items in the diary component, and make dietary recommendations. These dietary factors may be termed lifestyle choices that affect otitis media.

In an embodiment, the OMPM may communicate with other resources, such as servers connected to medical professionals, insurance companies, employers, and billing services. Typically, such communication will be wirelessly in the case of handheld devices, or through the internet in the case of desktop and laptop computers running the OMPM. Wireless connectivity (e.g. via Wi-Fi) with a router which provides Internet communication is also contemplated for any type of device.

In various embodiments, the OMPM may be provided as a public service that anyone can enroll in through a public registration system. In other embodiments, the OMPM may be provided as a closed system accessible only to members of a group, such as employees of a particular employer or health insurance members for a particular health insurance plan. For such groups, the OMPM may be a medical benefit. In other embodiments, the cost of the OMPM may be reimbursable by medical insurance companies or other health benefit providers.

1. A system for use by a user comprising a caregiver of a child, wherein the child is at risk for otitis media or suffering from otitis media, the system comprising
a. a computer;
b. a user interface operating on the computer, the user interface having a display screen and a keyboard;
c. a records management database stored in a non-volatile electronic medium operatively coupled to the computer;
d. a data management application operatively running on the computer and coupled to the user interface and;
e. wherein the records management database comprises data relevant to otitis media in the child; and
f. a diary of data entered by the user;
wherein the records management database contains information unique to a single child related to the child age, sex, height and weight, history of upper respiratory infections and/or otitis media and other medical issues;
wherein the user periodically enters data related to child's body temperature, degree of nasal discharge, appearance of pain or mood, or other factors relevant to acute otitis media into the records management database via the user interface on the initiative of the caregiver or in real time response to questions generated by the data management application and reminders to the caregiver to make specific observations of the child's health, child's body temperature, degree of nasal discharge, appearance of pain or mood, or other factors relevant to acute otitis media;
wherein the data management application is preprogrammed with information on the etiology and treatment of otitis media;
wherein the data management application is programmed to process data in the records management database and based on the results of the processed data to deliver pre-programmed messages, questions or recommendations to the caregiver in real time; and
wherein the messages and questions instruct or assist the caregiver in real time and provide information on lifestyle choices, dietary modifications to reduce mucus production, use of humidifiers or administration of medication and therapeutic modalities or instructions on when and how Galbreath technic should be performed or whether to consult a physician promptly that can be used by the caregiver for the prevention or treatment of otitis media in the child.

2. The system of claim 1, wherein the data management application provides reminders to the caregiver to perform the Galbreath technique on the child.

3. The system of claim 1, wherein the data management application prescribes the frequency that the Galbreath technique should be used on the child.

4. The system of claim 1, wherein the user interface operates on a computer platform selected from a website, a smartphone, a tablet computer, a desktop or laptop computer, and a wearable computer, or a combination thereof.

5. The system of claim 1, wherein the system transmits medical data or caregiver messages directly to a physician monitoring the health of the child.

6. The system of claim 1, wherein the system transmits medical data or caregiver messages directly to a physician monitoring the health of the child, and wherein the physician responds in real time with treatment options that can be performed by the caregiver.

7. The system of claim 1, wherein an account is created for the child, wherein the account includes a database unique to that child.

8. The system of claim 1, wherein the system can be used for a plurality of children wherein a records management database is created for each child, and wherein the records management database contains information unique to a single child.

9. A method for preventing or treating otitis media in a child at risk for otitis media or suffering from otitis media, comprising
a. a caregiver of a child;
b. a computer;
c. a user interface operating on the computer, the user interface having a display screen and a keyboard;
d. a records management database stored in a non-volatile electronic medium operatively coupled to the computer;
e. a data management application operatively running on the computer and coupled to the user interface and;
f. wherein the records management database comprises data relevant to otitis media in the child; and
g. a diary of data entered by the user;
wherein the user enters relevant data into the records management database via the user interface on the initiative of the caregiver or in response to questions generated by the data management application;
wherein the data management application is preprogrammed with information on the etiology and treatment of otitis media;
wherein the data management application is programmed to process data in the records management database and deliver pre-programmed messages or questions to the patient in real time; and
wherein the messages and questions instruct, or assist the caregiver in real time and provide information on lifestyle choices and therapeutic modalities that can be used by the caregiver for the prevention or treatment of otitis media in the child.

10. The method of claim 9, wherein the data management application provides reminders to the caregiver to perform the Galbreath technique on the child.

11. The method of claim 9, wherein the computer application is an application running on a mobile device, a desktop or laptop-type computer, or a website.

12. The method of claim 9, further comprising providing a summary of the patient's historic medical data for review by a physician.

13. A method for treating or preventing otitis media in a patient comprising
a. providing a computer application having a user interface, wherein the computer application stores data in one or more databases in computer memory;
b. providing a records management database stored in a non-volatile electronic medium operatively coupled to the computer;
c. providing a data management application coupled to the user interface and running on a computer;
d. receiving patient data into the database, wherein the data comprises medical parameters relevant to otitis media;
e. querying a caregiver via the user interface on a routine basis regarding observations and factors that affect otitis media, and receiving responses in real time from the patient that are stored in one or more of the databases; and
f. providing automated suggestions on a routine basis and in real time to the caregiver on factors and treatments affecting otitis media that are expected to prevent or treat otitis media in the patient.
